(19) United States
(12) Patent Application Publication
(10) Pub. No.: US 2022/0313591 A1
(43) Pub. Date: Oct. 6, 2022

(54) COMPOSITION COMPRISING BIOSURFACTANT AND CARBOXYBETAINE POLYMER

(71) Applicant: L'OREAL, PARIS (FR)

(72) Inventors: Hiroaki KAGA, Kanagawa (JP); Masanori ORITA, Kanagawa (JP); Sylvie BUN, Aulnay-sous-Bois (FR)

(21) Appl. No.: 17/618,204

(22) PCT Filed: Jun. 18, 2020
(86) PCT No.: PCT/JP2020/025116

§ 371 Date: Dec. 10, 2021

(30) Foreign Application Priority Data
Jun. 25, 2019 (JP) 2019-117471

(51) Int. Cl. A61K 8/81; A61Q 5/00; A61Q 19/00

(52) U.S. Cl. CPC A61K 8/8141; A61K 8/8147; A61Q 5/00; A61Q 19/00; A61K 2800/596

(57) ABSTRACT

The present invention relates to a composition comprising (a) at least one biosurfactant; and (b) at least one carboxybetaine polymer. The composition according to the present invention can provide a stable deposition of the composition on keratinous substances, such as skin, scalp, and hair.

### TECHNICAL FIELD

The present invention relates to a composition comprising at least one biosurfactant and at least one carboxybetaine polymer, preferably a cosmetic composition comprising the same.

### BACKGROUND ART

Biosurfactants are known to have unique bioactivities, such an anti-inflammation efficacy, anti-allergic efficacy, antibacterial efficacy, and the like. For example, sophorolipids and rhamnolipids are known to have anti-inflammation efficacy and potentially used on keratinous substances, such as skin.

Although biosurfactants have several unique bioactivities, there is a problem in that they are easily washed off from keratinous substances by, for instance, water or sweat, even if they are used in leave-on type cosmetic products.

Therefore, there is a need to provide a composition which can provide a stable deposition of biosurfactants on keratinous substances, such as skin, scalp, and hair, which is not easily removed from the keratinous substances by water or sweat.

### DISCLOSURE OF INVENTION

An objective of the present invention is to provide a composition which can provide a stable deposition on a substrate, such as keratinous substances, which is not easily washed off by water or sweat.

The above objective of the present invention can be achieved by a composition comprising:

(a) at least one biosurfactant; and
(b) at least one carboxybetaine polymer.

The (a) biosurfactant may be selected from rhamnolipids and sophorolipids.

The (b) carboxybetaine polymer may comprise at least one unit derived from a monomer represented by the following general formula (2):

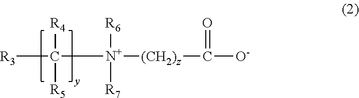

in which
R₃ indicates a polymerizable unsaturated group such as an acrylate, methacrylate, acrylamide or methacrylamide group;
y and z represent an integer from 1 to 5;
R₄ and R₅, which may be identical or different, represent a hydrogen atom or a linear or branched alkyl group comprising 1 to 5 carbon atoms; and
R₆ and R₇, which may be identical or different, represent a linear or branched alkyl group comprising 1 to 5 carbon atoms.

It is preferred that, in general formula (2),

R₃ indicates an acrylate or methacrylate group;
y and z represent an integer from 1 to 3, in particular 1 or 2;
R₄ and R₅, which may be identical or different, represent a hydrogen atom, methyl group, or ethyl group, in particular a hydrogen atom; and
R₆ and R₇, which may be identical or different, represent a methyl group or an ethyl group, in particular a methyl group.

The (b) carboxybetaine polymer may be selected from a copolymer of the carboxybetaine monomer and alkyl (meth)acrylate; a homopolymer of the carboxybetaine monomer; a copolymer of the carboxybetaine monomer, an alkyl (meth)acrylate quaternary ammonium salt, and polyoxyalkylene (meth)acrylate; and a copolymer of the carboxybetaine monomer, alkyl (meth)acrylate quaternary ammonium salt and hydroxyalkyl (meth)acrylate.

The amount of the (a) biosurfactant(s) in the composition may be 0.01% by weight or more, preferably 0.1% by weight or more, more preferably 0.5% by weight or more, and even more preferably 1% by weight or more; 15% by weight or less, preferably 10% by weight or less, more preferably 7.5% by weight or less, and even more preferably 5% by weight or less, relative to the total weight of the composition.

The amount of the (b) carboxybetaine polymer in the composition may be 0.001% by weight or more, preferably 0.005% by weight or more, and more preferably 0.01% by weight or more; 5% by weight or less, preferably 3% by weight or less, more preferably 2% by weight or less, and even more preferably 1% by weight or less, relative to the total weight of the composition.

The composition may comprise ionic surfactants, such as anionic surfactants, cationic surfactants, and amphoteric surfactants, other than the (a) biosurfactant(s) in an amount of 5% by weight or less, preferably 3% by weight or less, more preferably 1% by weight or less, and even more preferably 0.5% by weight or less, preferentially 0.1% by weight or less, in particular 0.01% by weight or less, relative to the total amount of the composition; or be free of ionic surfactants, such as anionic surfactants, cationic surfactants, and amphoteric surfactants, other than the (a) biosurfactant.

The composition may further include water in an amount of 60% by weight or more, preferably 70% by weight or more, and more preferably 80% by weight or more, relative to the total weight of the composition.

The composition may be a cleansing composition or a conditioner for keratinous substances, such as skin, scalp, lips, and hair.

The present invention also relates to a non-therapeutic cosmetic method for conditioning, caring, or cleansing of keratin substances, such as skin, scalp, lips, and hair, comprising:

applying onto the keratin substances a composition comprising:
(a) at least one biosurfactant; and
(b) at least one carboxybetaine polymer.

The present invention also relates to a use of a combination of

(a) at least one biosurfactant; and
(b) at least one carboxybetaine polymer
for conditioning, caring, or cleansing of keratin substances, such as skin, scalp, lips, and hair.

### BEST MODE FOR CARRYING OUT THE INVENTION

After diligent research, the inventors have discovered that a composition comprising (a) at least one biosurfactant and (b) at least one carboxybetaine polymer can provide a very stable deposition on a hydrophobic substrate having improved water tolerance, and then completed the present invention.

Thus, the composition according to the present invention is a composition comprising:

(a) at least one biosurfactant; and
(b) at least one carboxybetaine polymer.

The composition according to the present invention can provide a very stable deposition on a hydrophobic substrate having improved water tolerance. Therefore, the composition according to the present invention allows the deposition to stay on keratinous substances, such as skin, scalp, lips, and hair, for a long period. Accordingly, since the biosurfactant can provide a beneficial bioactivity on keratinous substances, such an anti-inflammation efficacy, anti-allergic efficacy, antibacterial efficacy, and the like, the composition according to the present invention is very suitable as a cosmetic composition, in particular as a cleansing composition or a conditioner for keratinous substances.

Hereinafter, the composition, process, and use according to the present invention will be explained in a more detailed manner.

### [Composition]

The composition according to the present invention comprises:

(a) at least one biosurfactant; and
(b) at least one carboxybetaine polymer.

The form of the composition according to the present invention is not particularly limited, as long as it is water based. The composition may take various forms, such as an aqueous gel, an aqueous solution, a gel, a lotion, a serum, a suspension, a dispersion, a fluid, a milk, a paste, a cream, an emulsion (O/W or W/O form), or the like. It is preferable that the composition according to the present invention be in the form of an aqueous solution or gel.

The composition according to the present invention may preferably be used as a cosmetic composition. Thus, the composition according to the present invention may be intended for application onto a keratin substance. Keratin substance here means a material containing keratin as a main constituent element, and examples thereof include the skin, scalp, nails, lips, hair, eyelashes, eyebrows, and the like. Thus, it is preferable that the composition according to the present invention be used for a cosmetic process for the keratin substance.

It is preferable that the composition according to the present invention be a cleansing composition or a conditioner for keratinous substances, such as skin, scalp, lips, or hair. For example, the composition can be a makeup remover, in particular a makeup remover for removing makeup from the keratin substance and a skin, scalp, lips, or hair care product.

The ingredients in the composition will be described in a detailed manner below.

### (Biosurfactant)

The composition according to the present invention comprises at least one (a) biosurfactant. If two or more (a) biosurfactants are used, they may be the same or different.

The term “biosurfactant” here means any substance which can function as a surfactant, and can be produced by microorganisms such as fungus and bacteria. The biosurfactant is a type of natural surfactant.

In one embodiment, the (a) biosurfactant may be selected from glycolipids.

The glycolipid biosurfactants may suitably be selected from rhamnolipids, glucolipids, trehalolipids, and mixtures thereof. Each will now be described in more detail below.

Rhamnolipids

These biosurfactants include a rhamnose moiety, and can be represented by general formula (I):

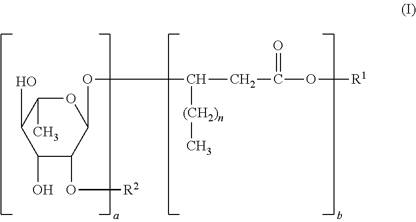

where
a is 1 or 2,
b is 1 or 2,
n is 4 to 10, preferably 6,
R¹ is H or a cation, preferably H, or a monovalent solubilizing cation,
R² is H or the group

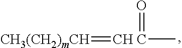

preferably H,
m is 4 to 10, and
the values of m and n need not be the same at each occurrence.

Rhamnolipids can be produced by bacterial fermentation. This is inherently advantageous in that products of bacterial fermentation can generally be derived from renewable raw materials and are likely to be biodegradable after use. Another advantage of the surfactants of formula (I) is that they can be produced as a by-product of enzyme manufacture.

Rhamnolipids can be produced by bacteria of the genus Pseudomonas. The bacterial fermentation typically utilizes as substrates a sugar or glycerol or an alkane or mixtures thereof.

Appropriate fermentation methods are reviewed in D. Haferburg, R. Hommel, R. Claus and H. P. Kleber in Adv Biochem. Eng./Biotechnol. (1986) 33, 53-90 and by F. Wagner, H. Bock and A. Kretschmar in Fermentation (ed. R. M. Lafferty) (1981), 181-192, Springer Verlag, Vienna.

Any sample of rhamnolipid will generally contain a variety of individual compounds within general formula (I). The proportions of individual compounds are governed by the microorganism species, and the particular strain employed for fermentation, the substrate materials supplied to the fermentation, and other fermentation conditions.

Bacterial fermentation generally produces compounds in which R¹ is hydrogen or a solubilizing cation. Such compounds can undergo conversion between the salt and the acid forms in aqueous solution, according to the pH of the solution. Common solubilizing cations are alkali metal, ammonium and alkanolamine.

As the rhamnolipids, for example, that sold under the name of RHEANCE One from EVONIK can be used.

Glucolipids

A second class of glycolipid biosurfactant in accordance with the present invention comprises glucolipids, which include a glucose moiety and can be represented by general formula (II):

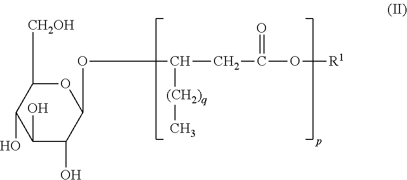

where
R¹ is H or a cation,
p is 1 to 4; and
q is 4 to 10, preferably 6.

Glucolipids can be produced by the bacterium Alcaligenes Sp.MM1. Appropriate fermentation methods are reviewed by M. Schmidt in his Ph.D. thesis (1990), Technical University of Braunschweig, and by Schulz et al. (1991) Z. Naturforsch 46C 197-203. The glucolipids are recovered from the fermentation broth via solvent extraction using ethyl ether or a mixture of either dichloromethane:methanol or chloroform:methanol.

Sophorolipids

A third class of glycolipid biosurfactant in accordance with the present invention comprises sophorolipids, which include a sophorose moiety and can be represented by general formula (III):

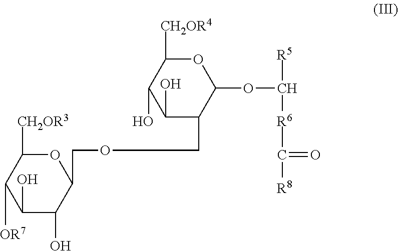

where
R³ and R⁴ are individually H or an acetyl group,
R⁵ is a saturated or unsaturated, hydroxylated or non-hydroxylated hydrocarbon group having 1 to 9 carbon atoms, preferably being a methyl group,
R⁶ is a saturated or unsaturated hydroxylated or non-hydroxylated hydrocarbon group having 1 to 19 carbon atoms,
with the proviso that the total number of carbon atoms in the groups R⁵ and R⁶ does not exceed 20 and is preferably from 14 to 18.

The sophorolipids may be incorporated into detergent compositions of the present invention as either the open chain free acid form, where R⁷ is H and R⁸ is OH, or in its lactone form, where a lactone ring is formed between R⁷ and R⁸ as shown by general formula (IV):

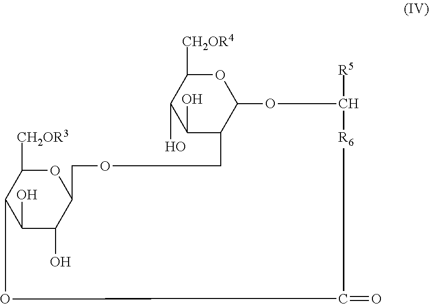

where
R³, R⁴, and R⁶ are as defined above,
with the proviso that at least one of R³ and R⁴ is an acetyl group.

Sophorolipids can be produced by yeast cells, for example Torulopsis apicola and Torulopsis bombicola. The fermentation process typically utilizes sugars and alkanes as substrates. Appropriate fermentation methods are reviewed in A. P. Tulloch, J. F. T. Spencer and P. A. J. Gorin, Can. J Chem (1962) 40 1326 and U. Gobbert, S. Lang and F. Wagner, Biotechnology Letters (1984) 6 (4), 225. The resultant product is a mixture of various open-chain sophorolipids and sophorolipid lactones, which may be utilized as mixtures, or the required form can be isolated. When the glycolipid biosurfactant comprises sophorolipids, the weight ratio of sophorolipids to additional surfactants is preferably in the range 4:1 to 3:2 and is more preferably 4:1.

As the sophorolipids, for example, that sold under the name of SOPHOLIANCE S from GIVAUDAN can be used.

Trehalolipids

A fourth class of glycolipid biosurfactant in accordance with the present invention comprises trehalolipids, which include a trehalose moiety and can be represented by general formula (V):

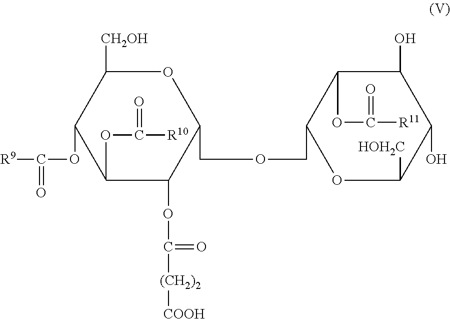

where
R⁹, R¹⁰, and R¹¹ are individually a saturated or unsaturated, hydroxylated or non-hydroxylated hydrocarbon of 5 to 13 carbon atoms.

Trehalolipids can be produced by bacteria fermentation using the marine bacterium Arthrobacter sp. Ek 1 or the fresh water bacterium Rhodococcus erythropolis. Appropriate fermentation methods are provided by Ishigami et al. (1987) J. Jpn Oil Chem Soc 36 847-851, Schultz et al. (1991), Z. Naturforsch 46C 197-203; and Passeri et al. (1991) Z. Naturforsch 46C 204-209.

Cellobioselipids

A fifth class of glycolipid biosurfactant in accordance with the present invention comprises cellobioselipids, which include a cellobiose moiety and can be represented by general formula (VI):

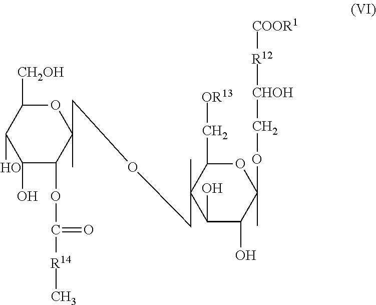

where
R¹ is H or a cation,
R¹² is a saturated or non-saturated, hydroxylated or non-hydroxylated hydrocarbon having 9 to 15 carbon atoms, preferably 13 carbon atoms,
R¹³ is H or an acetyl group; R¹⁴ is a saturated or non-saturated, hydroxylated or non-hydroxylated hydrocarbon having 4 to 16 carbon atoms.

Cellobioselipids can be produced by fungi cells from the genus Ustilago. Appropriate fermentation methods are provided by Frautz, Lang and Wagner (1986) Biotech Letts 8 757-762.

It may be preferable that the (b) biosurfactant be selected from rhamnolipids and sophorolipids.

In another embodiment, the (b) biosurfactant may be selected from surfactins and salts thereof.

The surfactins are natural surfactants produced by microorganisms from the genus Bacillus such as Bacillus subtilis IMA 1213, IAM 1069, JAM 1259, JAM 1260, IFO 3035 and ATCC 21332.

As the salts of surfactin, mention may be made of metal salts (e.g., salts with alkali metal such as sodium, potassium, and lithium, or alkali earth metal such as calcium and magnesium) and organic ammonium salts (e.g., salts with trimethylamine, triethylamine, tributylamine, monoethanolamine, diethanolamine, triethanolamine, lysine, and arginine) of a carboxylic group or groups of an amino acid or amino acids which form(s) surfactin.

The amount of the (a) biosurfactant(s) in the composition according to the present invention may be 0.01% by weight or more, preferably 0.1% by weight or more, more preferably 0.5% by weight or more, and even more preferably 1% by weight or more, relative to the total weight of the composition.

The amount of the (a) biosurfactant(s) in the composition according to the present invention may be 15% by weight or less, preferably 10% by weight or less, more preferably 7.5% by weight or less, and even more preferably 5% by weight or less, relative to the total weight of the composition.

### (Carboxybetaine Polymer)

The composition according to the present invention comprises (b) at least one carboxybetaine polymer. A single type of carboxybetaine polymer may be used, but two or more different types of carboxybetaine polymers may be used in combination.

The term “carboxybetaine polymer” here means a polymer comprising at least one carboxybetaine group, preferably at its side chain. The “carboxybetaine group” here means a group comprising a positively charged cationic moiety of a quaternary ammonium cation, and a negatively charged anionic moiety of a carboxylate, where no hydrogen atom is bound to the positively charged nitrogen in the quaternary ammonium cation and the quaternary ammonium cation is not adjacent to the carboxylate.

The carboxybetaine group in the polymer can be represented by the following general formula (1):

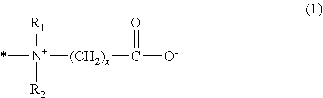

in which
R₁ and R₂, which may be identical or different, represent a linear or branched alkyl group comprising 1 to 5 carbon atoms;
x represents an integer from 1 to 5; and
* indicates an attachment point to a residual part of the polymer.

The carboxybetaine polymer may comprise at least one unit derived from a monomer comprising at least one carboxybetaine group. In the carboxybetaine polymer, a single type of carboxybetaine monomer may be used, but two or more different types of carboxybetaine monomers may be used in combination.

The carboxybetaine monomer is not particularly limited, but can be represented by the following general formula (2):

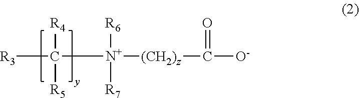

in which
R₃ indicates a polymerizable unsaturated group such as an acrylate, methacrylate, acrylamide or methacrylamide group;
y and z represent an integer from 1 to 5;
R₄ and R₅, which may be identical or different, represent a hydrogen atom or a linear or branched alkyl group comprising 1 to 5 carbon atoms; and
R₆ and R₇, which may be identical or different, represent a linear or branched alkyl group comprising 1 to 5 carbon atoms.

Preferably, in general formula (2),

R₃ indicates an acrylate or methacrylate group;
y and z represent an integer from 1 to 3, in particular 1 or 2;
R₄ and R₅, which may be identical or different, represent a hydrogen atom, methyl group, or ethyl group, in particular a hydrogen atom; and
R₆ and R₇, which may be identical or different, represent a methyl group or an ethyl group, in particular a methyl group.

It is preferable that the combination of the (a) biosurfactant(s) and the (b) carboxybetaine polymer can provide an improved efficiency of bioactivity with keratinous substances, such as skin, compared to a combination comprising other betaine polymers, such as a phosphobetaine polymer and sulfobetaine polymer instead of a carboxybetaine polymer. While the inventors do not wish to be bound by any particular theory, it is believed that the (b) carboxybetaine can provide a higher bioactivity, such as a higher skin or scalp care effect, together with the (a) biosurfactant(s), than other betaine polymers of phosphobetaine polymers and sulfobetaine polymers.

In addition, since the (b) carboxybetaine polymer bears a carboxylate group in its structure, it exhibits a higher pH-response property than phosphobetaine polymers and sulfobetaine polymers. Therefore, it can be expected that a certain property of the composition according to the present invention comprising the (b) carboxybetaine polymer can be modified by adjusting pH of the composition due to the higher pH-response property of the (b) carboxybetaine polymer.

The carboxybetaine polymer may further comprise another monomer unit derived from another monomer other than the carboxybetaine monomer.

The other monomer which can be comprised in the carboxybetaine polymer is not particularly limited, but includes for example, nonionic unsaturated monomers, anionic unsaturated monomers, cationic unsaturated monomers, and amphoteric unsaturated monomers, and a combination thereof.

The nonionic unsaturated monomers may include styrene monomers, such as styrene and hydroxy styrene, acrylate monomers, such as alkyl (meth)acrylate, hydroxyalkyl (meth)acrylate, alkoxyalkyl (meth)acrylate, polyoxyalkylene (meth)acrylate, N-alkyl (meth)acrylamide, (meth)acryloyl morpholine, diacetone (meth)acrylamide, acrylonitrile, and methyl itaconate, ethyl itaconate, vinyl acetate, vinyl propionate, N-vinylpyrrolidone, and N-vinylcaprolactam. A single type of another monomer or two or more different types of other monomers may be used in combination.

Said alkyl (meth)acrylate may include those comprising a C₁-C₁₈ alkyl group, such as methyl (meth)acrylate, n-butyl (meth)acrylate, isobutyl (meth)acrylate, t-butyl (meth)acrylate, 2-ethylhexyl (meth)acrylate, n-hexyl (meth)acrylate, n-octyl (meth)acrylate, cyclohexyl (meth)acrylate, decyl (meth)acrylate, undecyl (meth)acrylate, lauryl (meth)acrylate, tridecyl (meth)acrylate, myristyl (meth)acrylate, pentadecyl (meth)acrylate, palmityl (meth)acrylate, heptadecyl (meth)acrylate, stearyl (meth)acrylate, isostearyl (meth)acrylate, oleyl (meth)acrylate, and isobornyl (meth)acrylate. Said hydroxyalkyl (meth)acrylate may include those comprising a C₁-C₆ alkyl group, such as 2-hydroxyethyl (meth)acrylate, and 2-hydroxypropyl (meth)acrylate. Said alkoxyalkyl (meth)acrylate may include those comprising a C₁-C₆ alkyl group, such as ethoxyethyl (meth)acrylate and methoxyethyl (meth)acrylate. Said polyoxyalkylene (meth)acrylate may include those comprising C₁-C₄ polyoxyalkylene groups, such as polyethylene glycol mono(meth)acrylate and polypropylene glycol mono(meth)acrylate.

The anionic unsaturated monomers may include unsaturated monocarboxylic acids, such as (meth)acrylic acid and crotonic acid, unsaturated dicarboxylic acids, such as itaconic acid, maleic acid, fumaric acid, maleic anhydride, and citraconic acid, monoalkyl esters of unsaturated dicarboxylic acids, such as maleic acid monoalkyl ester, fumaric acid monoalkyl ester, and itaconic acid monoalkyl ester, sulfonic acid group containing unsaturated monomer and phosphoric acid group containing unsaturated monomer, and a combination thereof.

The cationic unsaturated monomers may include 1 to 3 amino group-containing unsaturated monomers and quaternary ammonium-containing unsaturated monomers.

Said 1 to 3 amino group-containing unsaturated monomers may include, for example, alkylaminoalkyl (meth)acrylate, such as aminoethyl (meth)acrylate, t-butylaminoethyl methacrylate, and methylaminoethyl (meth)acrylate, dialkylaminoalkyl (meth)acrylate, such as dimethylaminoethyl (meth)acrylate and diethylaminoethyl (meth)acrylate, dialkylaminoalkyl (meth)acrylamide, such as dimethylaminoethyl (meth)acrylamide and diethylaminoethyl (meth)acrylamide, and amino group-containing aromatic vinyl monomers, such as N, N-dimethylaminostyrene.

Said quaternary ammonium-containing unsaturated monomers may include, for example, alkyl (meth)acrylate quaternary ammonium salt, for example, those having a C₁-C₆ alkyl group, such as (meth)acryloyloxyethyl trimethyl ammonium chloride, (meth)acryloyl oxyethyl triethyl ammonium chloride, and (meth)acryloyl oxyethyl dimethyl benzyl ammonium chloride, and (meth)acryloyl oxyethyl methyl morpholino ammonium chloride, and alkyl (meth)acryloyl amide quaternary ammonium salt, such as (meta)acryloyl aminoethyl trimethyl ammonium chloride, (meth)acryloyl aminoethyl triethyl ammonium chloride, and (meth)acryloyl amino ethyl dimethyl benzyl ammonium chloride. The type of the salt of the quaternary ammonium-containing unsaturated monomers is not particularly limited, and may include a chloride salt, a fluoride salt, a bromide salt, and an iodide salt.

The amphoteric unsaturated monomers may include, for example, amine derivatives of (meth)acrylic acid, such as dimethylaminoethyl (meth)acrylate, dimethylaminopropyl (meth)acrylate, and diethylaminoethyl (meth)acrylate, and (meth)acrylamide derivatives such as dimethylaminoethyl (meth)acrylamide, and dimethylaminopropyl (meth)acrylamide.

Preferably, the monomer other than the carboxybetaine monomer is selected from alkyl (meth)acrylate, hydroxyalkyl (meth)acrylate, polyoxyalkylene (meth)acrylate, alkyl (meth)acrylate quaternary ammonium salt, and a mixture thereof.

In one preferred embodiment of the present invention, the (b) carboxybetaine polymer comprises at least one unit derived from alkyl (meth)acrylate quaternary ammonium salt.

In one preferred embodiment of the present invention, the (b) carboxybetaine polymer is selected from
a copolymer of the carboxybetaine monomer of general formula (2) and alkyl (meth)acrylate, such as a methacryloyl oxyethyl carboxybetaine-alkyl methacrylate copolymer sold under the name of YUKAFORMER R205, YUKAFORMER 301, YUKAFORMER SM, and YUKAFORMER 104D by MITSUBISHI CHEMICAL;
a homopolymer of the carboxybetaine monomer of general formula (2), such as polymethacryloyl ethyl betaine (INCI name: Polyquaternium-50) sold under the name of Plascize L-410 by GOO CHEMICAL;
a copolymer of the carboxybetaine monomer of general formula (2), alkyl (meth)acrylate quaternary ammonium salt, such as (meth)acryloyloxyethyl trimethyl ammonium chloride, and polyoxyalkylene (meth)acrylate, such as polyethylene glycol mono(meth)acrylate, in particular a copolymer of methacryloyl ethyl betaine, methacryloyl ethyl trimethyl ammonium chloride, and methoxy polyethylene glycol methacrylate (INCI name: Polyquaternium-49) sold under the name of Plascize L-440 by GOO CHEMICAL; and
a copolymer of the carboxybetaine monomer of general formula (2), alkyl (meth)acrylate quaternary ammonium salt, such as (meth)acryloyloxyethyl trimethyl ammonium chloride, and hydroxyalkyl (meth)acrylate, such as hydroxyalkyl (meth)acrylate comprising a C₁-C₆ alkyl group, in particular a copolymer of methacryloyl ethyl betaine, methacryloyl ethyl trimethyl ammonium chloride, and 2-hydroxyethyl methacrylate (INCI name: Polyquaternium-48) sold under the name of Plascize L-450 by GOO CHEMICAL.

The amount of the (b) carboxybetaine polymer(s) in the composition according to the present invention may be 0.001% by weight or more, preferably 0.005% by weight or more, and more preferably 0.01% by weight or more, relative to the total weight of the composition.

The amount of the (b) carboxybetaine polymer(s) in the composition according to the present invention may be 5% by weight or less, preferably 3% by weight or less, and more preferably 2% by weight or less, and even more preferably 1% by weight or less, relative to the total weight of the composition.

### (Fatty Alcohol)

The composition according to the present invention may further include at least one fatty alcohol. A single type of fatty alcohol may be included, but two or more different types of fatty alcohols may be included in combination.

The term “fatty alcohol” here means any saturated or unsaturated, linear or branched C₈-C₃₀ fatty alcohol, which is optionally substituted, in particular with one or more hydroxyl groups (in particular 1 to 4). If they are unsaturated, these compounds may comprise one to three conjugated or non-conjugated carbon-carbon double bonds.

Among the C₈-C₃₀ fatty alcohols, C₁₂-C₂₂ fatty alcohols, for example, are used. Mention may be made among these of lauryl alcohol, cetyl alcohol, stearyl alcohol, isostearyl alcohol, oleyl alcohol, behenyl alcohol, linoleyl alcohol, undecylenyl alcohol, palmitoleyl alcohol, linolenyl alcohol, myristyl alcohol, arachidonyl alcohol and erucyl alcohol, and mixtures thereof. In one embodiment, cetyl alcohol, stearyl alcohol or a mixture thereof (e.g., cetearyl alcohol), as well as myristyl alcohol, can be used as the fatty alcohol.

The amount of the fatty alcohol(s) in the composition according to the present invention may be 0.1% by weight or more, preferably 0.5% by weight or more, and more preferably 1% by weight or more; 20% by weight or less, preferably 15% by weight or less, and more preferably 10% by weight or less, relative to the total weight of the composition.

### (Non-Ionic Surfactant)

The composition according to the present invention may further include at least one nonionic surfactant. A single type of nonionic surfactant may be included, but two or more different types of nonionic surfactants may be included in combination.

As the nonionic surfactants, mention may be made of:
polyethoxylated fatty alcohols or polyglycerolated fatty alcohols, such as the adducts of ethylene oxide with lauryl alcohol, especially those containing from 9 to 50 oxyethylene units (Laureth-9 to Laureth-50, as the CTFA names); the adducts of ethylene oxide with behenyl alcohol, especially those containing from 9 to 50 oxyethylene units (Beheneth-9 to Beheneth-50, as the CTFA names); the adducts of ethylene oxide with cetyl alcohol, especially those containing from 10 to 30 oxyethylene units (Ceteth-10 to Ceteth-30, as the CTFA names); the adducts of ethylene oxide with stearyl alcohol, especially those containing from 2 to 20 oxyethylene units (Steareth-2 to Steareth-20, as the CTFA names); the adducts of ethylene oxide with oleyl alcohol, especially those containing from 10 to 30 oxyethylene units (Oleth-10 to Oleth-30, for example Oleth-20, as the CTFA names); and mixtures thereof
esters of sugar and of a C₈-C₂₄ fatty acid and their oxyalkylenated derivatives, such as polyethoxylated sorbitol esters of C₈-C₂₄ fatty acids, in particular Polysorbate 80, such as the product marketed under the name “TWEEN 80” by Croda; ethers of a sugar and of C₈-C₂₄ fatty alcohols, such as caprylyl/capryl glucoside such as the product marketed under the name “ORAMIX CG 110” by SEPPIC;
esters of fatty acids, especially of C₈-C₂₄ and preferably of C₁₆-C₂₂, and of polyol, especially of glycerol or sorbitol, such as glyceryl stearate, sold, for example, under the name Tegin M® by the company Goldschmidt, polyglyceryl diisostearate, polyglyceryl isostearate, polyglyceryl monostearate, diglyceryl tetraisostearate, polyethylene glycol diisostearate, polyglyceryl-10 pentastearate, glyceryl monooleate, glyceryl laurate, such as the product Imwitor 312® by the company Hills, diethylene glycol (di)laurate, decaglyceryl pentaoleate, decaglyceryl pentadiisostearate, glyceryl caprate/caprylate, polyglyceryl-2 (iso)stearate and (poly)ricinoleate;
oxyalkylenated alcohols, in particular oxyethylenated and/or oxypropylenated alcohols, which may comprise from 1 to 15 oxy ethylene and/or oxypropylene units, in particular ethoxylated C₈-C₂₄ and preferably C₁₂-C₁₈ fatty alcohols such as stearyl alcohol ethoxylated with 2 oxy ethylene units (CTFA name: Steareth-2 such as Brij 72 sold by the company Uniqema), or oxyethylenated oleyl alcohol.

The amount of the nonionic surfactant(s) in the composition according to the present invention may be 0.1% by weight or more, preferably 0.5% by weight or more, and more preferably 1% by weight or more; 10% by weight or less, preferably 5% by weight or less, and more preferably 3% by weight or less, relative to the total weight of the composition.

### (Polyol)

The composition according to the present invention may further comprise at least one polyol. A single type of polyol may be included, but two or more different types of polyols may be included in combination.

The term “polyol” here means an alcohol having two or more hydroxy groups. The polyol may be a C₂-C₁₂ polyol, preferably a C₂-C₉ polyol, comprising at least 2 hydroxy groups, and preferably 2 to 5 hydroxy groups. In particular, the polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, and 1,5-pentanediol.

The amount of the polyol(s) in the composition according to the present invention may be 1% by weight or more, preferably 2% by weight or more, and more preferably 5% by weight or more; 30% by weight or less, preferably 20% by weight or less, and more preferably 15% by weight or less, relative to the total weight of the composition.

### (Thickener)

The composition according to the present invention may further comprise at least one thickener. Two or more thickeners may be used in combination. Thus, a single type of thickener or a combination of different types of thickeners may be used.

The viscosity of the composition according to the present invention is not particularly limited. The viscosity can be measured at 25° C. with viscosimeters or rheometers preferably with cone-plane geometry. Preferably, the viscosity of the composition according to the present invention can range, for example, from 1 to 3000 Pa·s, and preferably from 1 to 2000 Pa·s at 25° C. and 1 s⁻¹.

The thickeners may be chosen from at least one of:
associative thickeners;
crosslinked acrylic acid homopolymers;
crosslinked copolymers of (meth)acrylic acid and of (C₁-C₆)alkyl acrylate;
nonionic homopolymers and copolymers comprising at least one of ethylenically unsaturated ester monomers and ethylenically unsaturated amide monomers;
ammonium acrylate homopolymers and copolymers of ammonium acrylate and of acrylamide;
polyvinylpyrrolidone,
polyvinyl alcohol,
modified or unmodified carboxyvinyl polymers, such as the products sold under the name Carbopol® (CTFA name: carbomer) by the company Goodrich;
polyacrylamides
polysaccharides; and
inorganic thickeners such as hydrophilic fumed silica, hydrophilic clays;
and mixtures thereof.

As used herein, the expression “associative thickener” means an amphiphilic thickener comprising both hydrophilic units and hydrophobic units, for example, comprising at least one C₈-C₃₀ fatty chain and at least one hydrophilic unit.

The amount of the thickener(s) in the composition according to the present invention may be 0.01% by weight or more, preferably 0.05% by weight or more, and more preferably 0.1% by weight or more; 5% by weight or less, preferably 3% by weight or less, and more preferably 1% by weight or less, relative to the total weight of the composition.

### (pH Adjusting Agent)

The pH of the composition according to the present invention may be adjusted to the desired value using acidifying or basifying agents, for example, which are commonly used in cosmetic products.

The pH of the composition according to the present invention may be 9.0 or less, more preferably 8.5 or less, and even more preferably 8.0 or less, and be 5.0 or more, more preferably 5.5 or more, and even more preferably 6.0 or more.

Among the acidifying agents, mention may be made, by way of example, of mineral or organic acids such as hydrochloric acid, ortho-phosphoric acid, sulfuric acid, carboxylic acids such as acetic acid, tartaric acid, citric acid, and lactic acid, and sulfonic acids.

Among the basifying agents, mention may be made, by way of example, of hydroxides of an alkali metal or an alkaline-earth metal, for instance sodium hydroxide or potassium hydroxide; quaternary ammonium hydroxides and guanidinium hydroxide; alkali metal silicates, such as sodium metasilicates; amino acids, preferably basic amino acids, such as arginine, lysine, ornithine, citrulline and histidine; carbonates and bicarbonates, particularly of a primary amine, secondary amine or tertiary amine, of an alkali metal or alkaline-earth metal, or of ammonium; and the compounds of the following formula:

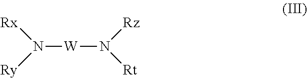

in which
W is a C₁-C₆ alkylene residue optionally substituted with a hydroxyl group or a C₁-C₆ alkyl group; Rx, Ry, Rz and Rt, which may be identical or different, represent a hydrogen atom or a C₁-C₆ alkyl, C₁-C₆ hydroxyalkyl or C₁-C₆ aminoalkyl group. Mention may especially be made of 1,3-diaminopropane, 1,3-diamino-2-propanol, spermine and spermidine.

The acidifying or basifying agent may be used in an amount ranging from 0.001% to 10% by weight, preferably from 0.01% to 5% by weight, and more preferably from 0.1% to 3% by weight, relative to the total weight of the composition.

### (Water)

The composition according to the present invention preferably includes water.

The amount of the water in the composition according to the present invention may be 60% by weight or more, preferably 70% by weight or more, and more preferably 80% by weight or more; relative to the total weight of the composition.

### (Optional Additives)

The composition according to the present invention may also comprise any optional additive(s) usually used in the field of cosmetics, chosen, for example, from anionic, cationic, nonionic or amphoteric polymers, oils, hydrophobic organic solvents, hydrophilic organic solvents, such as ethanol, gums, resins, dispersants, antioxidants, such as hydroxyacetophenon, film-forming agents, buffer such as potassium phosphate and dipotassium phosphate, preserving agents, such as salicylic acid, phenoxyethanol, and chlorphenesin, fragrances, neutralizers, antiseptics, UV-screening agents, cosmetic active agents such as vitamins, moisturizers, emollients or collagen-protecting agents, and mixtures thereof.

In one preferred embodiment of the present invention, the composition comprises:

(a) at least one biosurfactant selected from rhamnolipids and sophorolipids; and
(b) at least one carboxybetaine polymer selected from those composed of at least one carboxybetaine monomer and at least one monomer selected from alkyl (meth)acrylate, hydroxyalkyl (meth)acrylate, polyoxyalkylene (meth)acrylate, alkyl (meth)acrylate quaternary ammonium salt, and a mixture thereof.

In another preferred embodiment of the present invention, the composition comprises ionic surfactants, such as anionic surfactants, cationic surfactants, and amphoteric surfactants, other than the (a) biosurfactant(s), in an amount of 5% by weight or less, preferably 3% by weight or less, more preferably 1% by weight or less, and even more preferably 0.5% by weight or less, preferentially 0.1% by weight or less, in particular 0.01% by weight or less, relative to the total amount of the composition.

In another preferred embodiment of the present invention, the composition is free of ionic surfactants, such as anionic surfactants, cationic surfactants, and amphoteric surfactants, other than the (a) biosurfactant(s).

The composition according to the present invention can be prepared by mixing the above-described essential and optional ingredients in a conventional manner. In the case that at least one of the above ingredients is solid at room temperature, the ingredient can be heated until it is dissolved.

For example, the composition according to the present invention can be prepared by a process comprising the step of mixing

(a) at least one biosurfactant; and
(b) at least one carboxybetaine polymer.

It is possible to further comprise mixing any of the optional ingredients and heating the composition until an ingredient is dissolved.

### [Method and Use]

The present invention also relates to a non-therapeutic method or process, preferably a cosmetic method or process, and more preferably a cosmetic method or process for conditioning, caring or cleansing keratin substances, such as skin, scalp, lips, and hair, comprising:

applying onto the keratin materials a composition comprising:
(a) at least one biosurfactant; and
(b) at least one carboxybetaine polymer.

The present invention also relates to a use of a combination of

(a) at least one biosurfactant; and
(b) at least one carboxybetaine polymer
for conditioning, caring or cleansing keratin substances, such as skin, scalp, lips, and hair.

The keratin materials may be selected from skin, scalp, hair, lips, nails, eyelashes, and eyebrows.

The composition used in the process and use according to the present invention may include any of the optional ingredients as explained above for the composition according to the present invention.

While the inventors do not wish to be bound by any particular theory, it is believed that the (b) carboxybetaine can support the (a) biosurfactant(s) to stay on a hydrophobic substrate, such as keratinous substances, due to its specific ionic charge. This means that it is believed that the (b) carboxybetaine can enhance a water tolerance of the (a) biosurfactant(s) on a hydrophobic substrate, such as keratinous substances.

Therefore, the present invention may also relate to a use of the (b) carboxybetaine polymer for enhancing a water tolerance of a deposition of the (a) biosurfactant(s) on a hydrophobic substrate, such as keratinous substances, in particular skin, scalp, lip, and hair. This use can provide improved cosmetic effects on the keratinous substances due to the bioactivity produced from the (a) biosurfactant(s).

### Examples

The present invention will be described in more detail by way of examples which however should not be construed as limiting the scope of the present invention.

### [Lotion Composition]

Lotion compositions according to Examples 1 and 2 (Exs. 1 and 2) and Comparative Examples 1 to 3 (Comp. Exs. 1 to 3) were prepared. The formulations are shown in Table 1. The numerical values for the amounts of the components shown in Table 1 are all based on “% by weight” as active raw materials. Sophorolipid was obtained from GIVAUDAN (trade name: SOPHOLIANCE S), Rhamnolipid was obtained from EVONIK (trade name: RHEANCE One), and Methacryloyl Ethyl Betaine/Acrylates Copolymer was obtained from MITSUBISHI CHEMICAL (trade name: YUKAFORMER R205).

These compositions were prepared by first mixing sophorolipid or rhamnolipid, if present, water, potassium hydroxide, potassium phosphate, and dipotassium phosphate under ambient environment (1 atm, 25° C.) to prepare a homogenous mixture and then mixing ethanol and methacryloyl ethyl betaine/acrylates copolymer, if present, with the mixture until it became even.

### [Evaluation]

### (Stability of Deposition)

The stability of the deposition according to each of the compositions according to Examples 1 and 2 and Comparative Examples 1 to 3 was evaluated as below. 100 μL of sample of each of the compositions was applied on a 1-cm² area of a PMMA substrate and then dried at 40° C. A contact angle of 1 μL of water and the PMMA area, where the sample was applied, was measured with an optical contact angle measuring and contour analysis system, OCA-15 plus from DATA Physics Instruments GmbH, after 3 minutes once the water was placed on the area and after washing the area 1 to 3 times with 1 mL of water. The right and left sides of contact angles of the water droplet were obtained by software and the average was calculated. A lower contact angle represents the point that a greater amount of the deposition of the bio surfactants remained on the PMMA surface.

The average evaluation results are summarized in Table 1 below. “DL” in Table 1 stands for detection limit which means the angle was too low to measure it.

TABLE 1

| | | | | Comp. | Comp. | Comp. |
|---|---|---|---|---|---|---|
| Ingredients | | Ex. 1 | Ex. 2 | Ex. 1 | Ex. 2 | Ex. 3 |
| Water | | QS | QS | QS 100 | QS 100 | QS 100 |
| | | 100 | 100 | | | |
| Potassium Hydroxide | | QS | QS | QS pH7 | QS pH7 | QS pH7 |
| | | pH7 | pH7 | | | |
| Potassium Phosphate | | 0.036 | 0.036 | 0.036 | 0.036 | 0.036 |
| Dipotassium | | 0.064 | 0.064 | 0.064 | 0.064 | 0.064 |
| Phosphate | | | | | | |
| Sophorolipid | | 2 | — | — | 2 | — |
| Rhamnolipid | | — | 2 | — | — | 2 |
| Ethanol | | 0.07 | 0.07 | 0.07 | — | — |
| Methacryloyl Ethyl | | 0.03 | 0.03 | 0.03 | — | — |
| Betaine/Acrylates | | | | | | |
| Copolymer | | | | | | |
| Evaluation | | | | | | |
| Stability of | No | DL | DL | DL | DL | DL |
| Deposition | Wash | | | | | |
| | Contact | DL | DL | 19.60 | DL | DL |
| | Angle | | | | | |
| | After 1 | | | | | |
| | Wash | | | | | |
| | Contact | DL | DL | 35.95 | 32.15 | 35.70 |
| | Angle | | | | | |
| | After 2 | | | | | |
| | Washes | | | | | |
| | Contact | DL | DL | 62 | 39.9 | 42.05 |
| | Angle | | | | | |
| | After 3 | | | | | |
| | Washes | | | | | |

As can be seen from Table 1, the PMMA substrate treated with the compositions according to Examples 1 and 2, which include the specific combination of the ingredients of the (a) biosurfactant and the (b) carboxybetaine polymer of the present invention, exhibited undetectable low contact angle with water, even after it was washed by water for three times. On the other hand, the composition according to each of Comparative Examples 1 to 3, which lack the biosurfactant, carboxybetaine polymer, or both, showed inferior stability of the deposition. Therefore, it can be said that the specific combination of the present invention can provide very stable deposition on the substrate having improved water tolerance.

### [Gel Composition]

Gel compositions according to Example 3 (Ex. 3) and Comparative Example 4 (Comp. Ex. 4) were prepared. The formulations are shown in Table 2. The numerical values for the amounts of the components shown in Table 2 are all based on “% by weight” as active raw materials.

These compositions were prepared by first mixing sophorolipid, water, cetyl alcohol, stearyl alcohol, glyceryl stearate, glycerin, and arginine evenly at 85° C. to prepare a homogeneous mixture. In addition carbomer, arginine, and water were mixed at 85° C. to prepare another homogeneous mixture, and these mixtures were combined. The obtained mixture was cooled to room temperature (25° C.) and then pentylene glycol, phenoxyethanol, chlorphenesin, ethanol (Example 3), and methacryloyl ethyl betaine/acrylates copolymer (Example 3) were added to the mixture.

The mixture was mixed until it became even. The total amount of arginine used was 0.5% by weight.

### [Evaluation]

### (Sensory Assessment)

The feeling of deposition of the composition according to each of Example 3 and Comparative Example 4 were tested by 6 sensory panel specialists. About 3 g of the composition was applied on their hands and rinsed off with water, and then the sense of touch was evaluated. The sense of touch was scored by the following criteria.

4: strong deposition feeling
3: intermediate deposition feeling
2: weak deposition feeling
1: no deposition feeling

The average scores of the evaluation are summarized in Table 2 below.

TABLE 2

| Ingredients | Ex. 3 | Comp. Ex. 4 |
|---|---|---|
| Sophorolipid | 2 | 2 |
| Cetyl Alcohol | 1.95 | 1.95 |
| Stearyl Alcohol | 1.3 | 1.3 |
| Glyceryl Stearate | 1.75 | 1.75 |
| Glycerin | 10 | 10 |
| Water | QS 100 | QS 100 |
| Arginine | QS pH7 | QS pH7 |
| Carbomer | 0.196 | 0.196 |
| Pentylene Glycol | 1 | 1 |
| Phenoxyethanol | 0.5 | 0.5 |
| Chlorphenesin | 0.28 | 0.28 |
| Ethanol | 1.4 | — |
| Methacryloyl Ethyl | 0.6 | — |
| Betaine/Acrylates | | |
| Copolymer | | |
| Evaluation | | |
| Score | 2.67 | 1.8 |

As can be seen from Table 2, the compositions according to Example 3, which includes the specific combination of the ingredients of the (a) biosurfactant and the (b) carboxybetaine polymer of the present invention, exhibited a higher score of sense of touch, which means that the specialists felt that a greater amount of the deposition remained on their hands even after they washed their hands with water. On the other hand, the composition according to Comparative Example 4, which lacks carboxybetaine polymer, showed a lower score.

Therefore, it can be said that the specific combination-of the present invention can provide a very stable deposition on the substrate having improved water tolerance. This means that the composition according to the present invention allows the deposition to stay on the keratinous substance for a long period. Accordingly, it can be concluded that the composition according to the present invention is very suitable as a cosmetic composition, in particular as a cleansing composition or a conditioner for keratinous substances, since the biosurfactant can provide a beneficial bioactivity on keratinous substances.

1. A composition comprising:
(a) at least one biosurfactant; and
(b) at least one carboxybetaine polymer.

2. The composition according to claim 1, wherein the (a) biosurfactant is selected from rhamnolipids and sophorolipids.

3. The composition according to claim 1, wherein the (b) carboxybetaine polymer comprises at least one unit derived from a monomer represented by the following general formula (2): 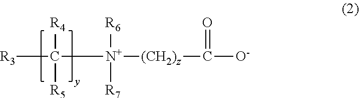
in which
R₃ indicates a polymerizable unsaturated group;
y and z represent an integer from 1 to 5;
R₄ and R₅, which may be identical or different, represent a hydrogen atom or a linear or branched alkyl group comprising 1 to 5 carbon atoms; and
R₆ and R₇, which may be identical or different, represent a linear or branched alkyl group comprising 1 to 5 carbon atoms.

4. The composition according to claim 3, wherein in the general formula (2),
R₃ indicates an acrylate or methacrylate group;
y and z represent an integer from 1 to 3;
R₄ and R₅, which may be identical or different, represent a hydrogen atom, methyl group, or ethyl group; and
R₆ and R₇, which may be identical or different, represent a methyl group or an ethyl group.

5. The composition according to claim 1, wherein the (b) carboxybetaine polymer is selected from a copolymer of carboxybetaine monomers and alkyl (meth)acrylate; a homopolymer of carboxybetaine monomers; a copolymer of carboxybetaine monomers, alkyl (meth)acrylate quaternary ammonium salt, and polyoxyalkylene (meth)acrylate; and a copolymer of carboxybetaine monomers, alkyl (meth)acrylate quaternary ammonium salt and hydroxyalkyl (meth)acrylate.

6. The composition according to claim 1, wherein the amount of the (a) biosurfactant(s) in the composition is 0.01% by weight or more and 15% by weight or less, relative to the total weight of the composition.

7. The composition according to claim 1, wherein the amount of the (b) nonionic surfactant(s) in the composition ranges from 0.1 to 30% by weight relative to the total weight of the composition.

8. The composition according to claim 1, wherein the amount of the (b) carboxybetaine polymer in the composition is 0.001% by weight or more and 5% by weight or less relative to the total weight of the composition.

9. The composition according to claim 1, wherein the composition comprises ionic surfactants other than the (a) biosurfactant(s) in an amount of 5% by weight or less relative to the total amount of the composition; or be free of ionic surfactants other than the (a) biosurfactant.

10. The composition according to claim 1, wherein the composition further includes water in an amount of 60% by weight or more relative to the total weight of the composition.

11. The composition according to claim 1, wherein the composition is a cleansing composition or a conditioner for keratinous substances.

12. A non-therapeutic cosmetic method for conditioning, caring, or cleansing keratin substances, comprising:
applying onto the keratin substances a composition comprising:
(a) at least one biosurfactant; and
(b) at least one carboxybetaine polymer.

13. A method comprising:
conditioning, caring, or cleansing keratinous substances by applying onto the keratin substances a combination of:
(a) at least one biosurfactant; and
(b) at least one carboxybetaine polymer.
